(19) United States
(12) Patent Application Publication
(10) Pub. No.: US 2005/0177030 A1
(43) Pub. Date: Aug. 11, 2005

(54) Physiological, DNA identification security monitoring, and response system

(76) Inventors: Scott Ponquinette, Hampton, VA (US); Willie W. Truitt, Zuni, VA (US)

Correspondence Address: Bradley D. Goldizen;Suite 102, 505 So. Independence Blvd., Virginia Beach, VA, 23452, US

(21) Appl. No.: 10/775,117

(22) Filed: Feb. 11, 2004

(51) Int. Cl. A61B 5/00

(52) U.S. Cl. 600/300

(57) ABSTRACT

A biological, DNA, security and medical alert system includes a remote monitoring unit worn by a user. The remote monitoring unit includes biosensors that monitor biorhythms of the user, an audio and video device for communication, a microprocessor monitors data collected by the biosensors to detect anomalies indicative of a medical condition, alerts a central unit, and automatically, manually and or via voice control administers medications stored in the remote unit.

### BACKGROUND OF THE INVENTION

The present invention generally relates to a monitoring security system. That through a voice activated and recognition device causes at least (1) one of the needles in the unit to puncture the wearers skin and takes a sample of the blood (DNA) and records the results in the data Permanente memory. At least (1) one of these micro physiological sensors will simultaneously take and record the physiological functions of the wearer and also enter the results into the Permanente memory record.

This initial voice, DNA and physiological record is now a secured way to identify the wearer and may be used not only to monitor the biorhythms and biological functions of the wearer but can also be used at security check points such as military (ID), airports, train stations, boat docks, secured areas, buildings, identification for credit loans, hospitals, schools and any other place that requires a definite identification that cannot be easily duplicated. Each wearer can be pre-coded after a background check to clear them as possible non-terrorists for traveling and etc, because their physiological data and DNA is different from others.

Also this monitoring system is perfect for storing and retrieving pertinent records; such as social security information, health records, family ancestry, especially the ability to record their photographs, verbal speech, as well as their written records. Each level of records can only be retrieved by a special code on a need to know basis, all units will have universal cords with special adaptable ends that can plug into most computer systems so the user can enter as well as retrieve information.

More specifically, the system is directed towards detecting and providing an emergency response to a specific medical condition of a wearer. An individual, having a medical condition such as heart disease or diabetes, wears a remote unit. The remote unit includes biosensors that detect biorhythms such as temperature, blood sugar level, and pulse rate, as well as at least one type of medication for treating the condition stored within the remote unit.

Many times individuals suffering from dangerous medical conditions such as a heart attack or insulin shock are incapacitated and incapable of administering medication to themselves or alerting others of these dangerous medical conditions. It is known that several types of medicines may save the life of one suffering from a heart attack or insulin shock, but only if the medicine is administered in a timely fashion.

The system comprises a remote monitoring unit that includes sensors for collecting and recording physiological data and DNA data. A microprocessor compares real time data with stored base line data. The remote monitoring unit communicates with a central control unit to relay data and control information between the two. Either may control the injection of a medication within the remote monitoring unit.

### SUMMARY OF THE INVENTION

A monitoring system of the present type comprises a remote unit worn by an individual and a central unit that receives real time or near real time biological data from the remote unit. The remote unit observes and records or reports the biorhythms of a wearer's biological functions to the central unit. When a medical condition arises, the individual exhibits certain physiological characteristics. For example, a person having a heart attack may experience profuse sweating, a racing pulse or palpitations, and nausea. Thus, when the system recognizes one or more of these symptoms, an emergency medical condition is realized and appropriate response steps are taken to assist medical personnel in arriving at the scene of the individual.

Sensors that are located in or connected to the remote unit detect and monitor physiological characteristics such as temperature, blood sugar level, and pulse rate.

A microprocessor within the remote unit collects, stores, and analyzes data collected from the sensors. For example, the microprocessor collects and stores base-line data while the wearer conducts ordinary activities. The microprocessor then periodically collects data from the sensors and compares the collected data with the recorded base-line data. When experiencing an adverse medical condition, the collected data deviates greatly from the base-line data, thereby indicating the existence of the medical condition.

Upon detecting that the wearer is experiencing an adverse medical condition, for example a heart attack or insulin shock, the remote unit alerts a central monitoring unit of the medical condition while administering a dose of medication to the wearer. The remote unit may also continue to transmit pertinent biological information to the central monitoring unit until a first responder arrives at the scene to administer further medical attention.

Other embodiments of the invention include an audible alarm feature to alert others nearby of the medical condition. A self-test feature may be included for assessing whether the remote unit and system is operating properly. The remote monitoring unit may comprise a global positioning receiver for relaying location of the remote unit to the central monitoring unit to assist in directing first responders to the wearer. A retransmission unit connected to a telephone or computer network relays information to and from the remote monitoring unit.

The remote monitoring unit and the central monitoring unit may include a power supply that comprises photovoltaic cells for producing electricity from solar energy, batteries or other known electrical sources both alternating and direct current.

It is an object of the invention to provide a lightweight wireless monitoring device for detecting at least one medical condition of a wearer. The wireless monitoring device comprises a microprocessor for collecting and analyzing data.

It is a further object of the invention to provide a medication application device that is activated manually or through an electronic means to inject a medication into a wearer while he is experiencing an adverse medical condition.

It is another object of the invention to provide a monitoring system that stores base-line data relating to an individual's biorhythms for comparison with real time data to determine when an adverse medical condition exists.

It is a further object of the invention to provide a remote monitoring device for storing base-line data and real time data relating to the biological functions of the wearer.

It is another object of the invention to provide a memory for storing a routine that compares the base-line data and real time data relating to the biological functions of the wearer to create an activation signal when detected data indicates that a medical condition exists.

These and other objects of the invention and advantages of the invention will be set forth, appear in part or become apparent after considering the specification and accompanying drawings. It is to be realized that the following embodiments of the invention have been represented in their simplest form for ease in understanding the invention.

### BRIEF DESCRIPTION OF THE DRAWINGS

FIG. 1 is a block diagram of the system of the present invention.

FIG. 2 is a simplified block diagram of the remote monitoring unit.

FIG. 3 is a perspective plan view of the remote monitoring unit.

FIG. 4 is a cross-section elevation view of the remote monitoring unit showing a micro-needle and taken from line A-A of FIG. 3.

FIGS. 5A and 5B are perspective views of the needle shown in different positions.

FIG. 6 is a perspective view of the medication capsule piercing device.

FIG. 7 is a flowchart of the preferred monitoring process of the remote monitoring unit.

FIG. 8 is a flowchart of the preferred monitoring process of the central monitoring unit.

### DETAILED DESCRIPTION OF THE INVENTION

The present invention is a system comprised of a central monitoring unit and a remote unit worn by a user. The remote unit samples certain types of physiological data for comparison with a stored base-line data. When the sampled data exceeds a certain threshold, an emergency medical condition exists that is detected by the remote unit. In one embodiment, the remote monitoring unit includes a process for detecting when an emergency condition exists and providing a response by automatically injecting the user with a medicine for treating the medical condition. In another embodiment, the system includes a central monitoring unit that controls an injection of medication in response to detecting a medical condition.

When the remote unit exceeds a certain distance from the re-transmitter, the remote unit stores physiological data and may assume a mode wherein it controls the injection of a dose of medication in response to detecting a medical condition. Thus, the remote unit may advantageously use less power to achieve the same protective results when near a re-transmitter. Therefore, the remote unit will yield control of the injection process to the control monitoring unit when near the re-transmitter.

The monitoring system that monitors physiological data of a wearer includes a central monitoring unit and a remote monitoring unit. The remote monitoring unit comprises a microprocessor that compares collected real-time physiological data with stored base-line physiological data to determine the existence of a medical condition of a wearer. A memory includes instructions for collecting and storing the real-time and base-line physiological data relating to a health condition of a wearer. A display connected to the microprocessor displays instructions for use of the remote monitoring unit as well as medical instructions.

At least one sensor connects to the microprocessor and periodically samples the physiological data. A medication storage compartment stores a medication to be administered in response to a detected medical condition. An injection device connected to the medication storage compartment delivers an injection of the medication to the wearer when the medical condition is detected. A communications device transmits collected physiological data from the remote monitoring unit to the central monitoring unit.

The monitoring system may also comprise a global positioning system receiver that detects a location of the remote monitoring unit, such that the location of the remote monitoring unit may be relayed to the central monitoring unit. In addition, the communications device includes a speaker and microphone for providing audio communications between the wearer and an operator located at the central monitoring unit. In another embodiment, the communications device includes a speaker, microphone and camera for providing audio and video communications between the wearer and an operator located at the central monitoring unit.

A needle compartment for housing the injection device includes an antiseptic film having a side exposed to the injection device and an opposite side exposed to a surface of a wearer's skin such that the injection device penetrates and punctures the antiseptic film when the medication is administered. The injection device may include a solenoid controlled by the microprocessor for remotely administering the medication. Alternatively, the injection device may include a piezoelectric member for driving the injection device. On the other hand, the injection device may include a magnetostrictive member that drives the injection device. The medication storage compartment comprises a hinged cover connected to the casing of the remote monitoring unit.

In a further embodiment, the remote monitoring unit comprises a speaker that alerts nearby individuals to a medical condition of a wearer when the wearer is unconscious. A re-transmitter unit may relay information and data to and from the remote monitoring unit from and to the central monitoring unit.

In another embodiment, a medical condition monitoring system includes a central monitoring unit and a plurality of remote monitoring units. The system comprises the central monitoring unit that includes a transceiver for communicating with the remote monitoring units. A microprocessor within the central monitoring unit stores physiological data of a plurality of wearers. A display screen displays information from a selected remote monitoring unit. A microprocessor located within each remote monitoring unit compares collected real time physiological data with stored base-line physiological data to determine the existence of a medical condition of a wearer. A memory within each remote monitoring unit includes instructions for collecting and storing real time and base-line physiological data relating to a health condition of a wearer. A display connected to the microprocessor located within each remote monitoring unit displays instructions for use of the remote monitoring unit. At least one sensor located within each remote monitoring unit and connected to the microprocessor periodically samples physiological data of a wearer. A medication storage compartment located within each remote monitoring unit stores a medication to be administered in response to a detected medical condition. An injection device located within each remote monitoring unit and connected to the medication storage compartment delivers an injection of the medication to the wearer when the medical condition is detected. A communications device located within each remote monitoring unit transmits collected physiological data from the remote monitoring unit to the central monitoring unit.

Each remote monitoring unit includes a unique identification code. The identification code is used by a microprocessor in the system for accepting operating instructions. This prevents multiple remote monitoring units from injecting individuals who are not experiencing an adverse medical condition from being inadvertently injected should one of the remote monitoring units indicate the presence of a medical condition. It may also be used for military applications to sedate a captured soldier to prevent him from disclosing vital information. The global positioning system receiver can then be used to determine the location of the soldier for extrication.

A process for monitoring a wearer of a health monitoring device comprises collecting data during normal activities and when a wearer is not experiencing a serious medical condition. The data is stored as base-line data to be compared with real time data such that the existence of a medical condition is recognized when the real time data exceeds an acceptable threshold. An injection is then administering when the medical condition is recognized. The process may further include confirming consciousness of a wearer to determine whether an injection should be automatically administered. A list of medical instructions may be relayed from the central monitoring unit to the wearer. Medical authorities may be alerted when a medical condition has been determined.

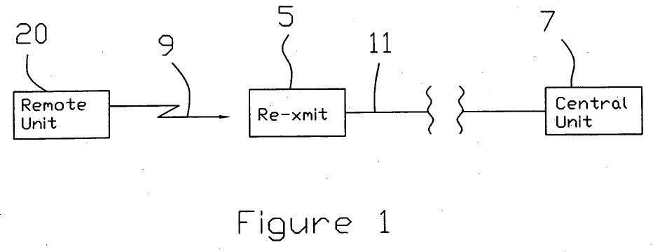

FIG. 1 is a block diagram of a physiological monitoring system of the present invention. The system comprises a remote unit 20 as mentioned previously. The remote unit 20, using radio frequencies or other wireless signals 9, communicates with a re-transmitter 5 connected to a telephone or computer network 11. The re-transmitter 5 relays data from the remote unit 20 to a central monitoring unit 7. Instructions for controlling the remote unit 20 are relayed from the central monitoring unit 5 through the re-transmitter.

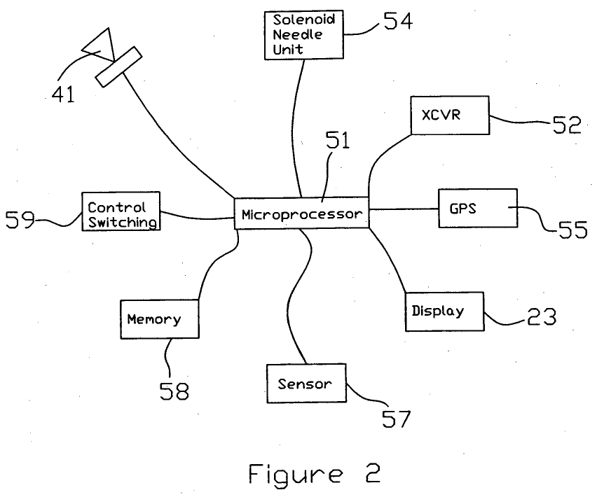

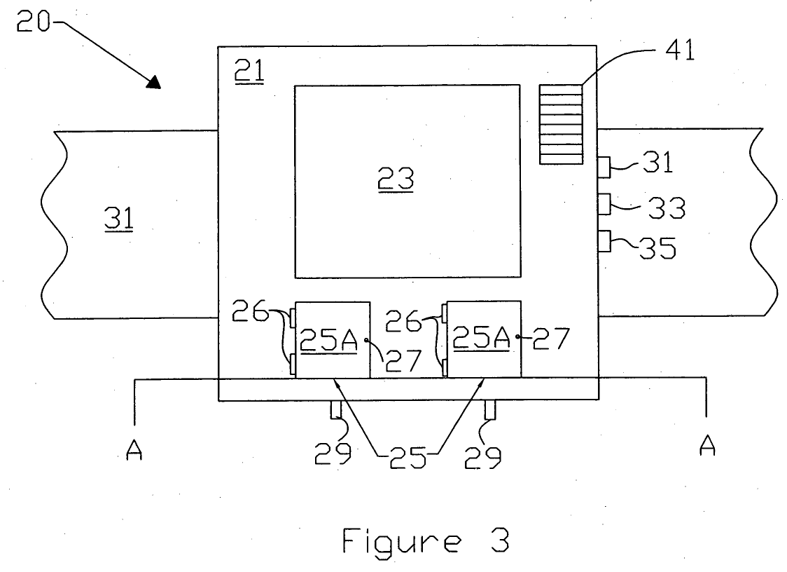

FIGS. 2 and 3 are a block diagram and a perspective view of the remote monitoring unit 20, respectively. The remote monitoring unit 20 comprises a microprocessor 51 for controlling the collecting, analyzing, and transmitting of physiological data collected from at least one sensor 57, as well as control of the remote monitoring unit 20. A transceiver 52 wirelessly transmits data from the microprocessor 51 to the re-transmitter 5 and receives instructions therefrom. The microprocessor 51 also controls the administering of medication through a solenoid needle unit 54 or other such electrically actuated device.

A speaker 41 emits audible signals for alerting nearby individuals of the existence of a medical condition as well as providing first aid instructions. The remote monitoring unit 20 may be equipped with a microphone for establishing an audio link between the remote monitoring unit 20 and the central monitoring units. Likewise, the remote monitoring unit may also include a camera and video capabilities for capturing a picture of the wearer. Pictures may be displayed on a display screen 23 for assisting in the administering of first aid or to confirm the existence of a medical condition. Control switching 59 such as select, scroll up and scroll down buttons allow the user to scroll through a menu to select a plurality of functions on the display screen 23. The remote monitoring unit 20 may also include a global positioning system receiver 55 for determining the location of the remote unit 20 and transmitting data relating to such location to the central monitoring unit 7.

A memory 58 is provided for storing operating instructions as well as temporarily storing collected data. In one embodiment, the memory may store base-line data for comparing the collected real time data to determine when a medical condition exists. It should be noted that the memory 58 might be included in the microprocessor 51.

FIG. 3 depicts the remote monitoring unit 20 in the preferred embodiment. A case or housing 21 secures the various components that comprise the remote monitoring unit 20. A band 31, such as a watchband, holds the remote monitoring unit 20 in contact with the skin of the user. A sensor 57 may be positioned on the underside of the remote monitoring unit 20, or alternatively, it may be positioned along the band 31. The sensor 57 connects to the microprocessor for relaying data thereto.

The remote monitoring unit 20 includes at least one medication storage compartment 25. However, FIG. 3 shows two medication storage compartments 25. Each medication storage compartment 25 includes a cover 25A. A hinge 26 couples the cover 25A to the casing 21. A closure device 27, such as a small screw, secures the cover 25A in a closed position. The hinged cover allows the replacement of a spent medication capsule. Each medication storage compartment 25 is equipped with a manual medication activation button 29 for manually injecting the user. Control switches 31, 33 and 35 allow the user to scroll through and select options displayed on display screen 23. The control switches may include a select button 31, a scroll-up button 33 and a scroll-down button 35 or other known variations thereof.

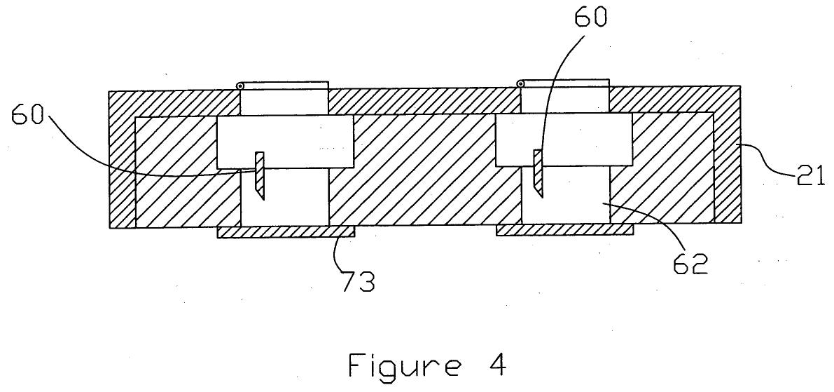

FIG. 4 is a cross-section elevation view of the remote monitoring unit showing a micro-needle 60 and taken from line A-A of FIG. 3. For ease in understanding the invention, all internal components of the remote monitoring unit with the exception of the needles 60 have been removed from FIG. 3. The needles 60 are each stored in a separate needle compartment 62 that includes an antiseptic film 73. When activated, the needle 62 punctures the antiseptic film 73 to sterilize the needle 62 prior to penetrating the skin of the user. This assures that the needles are not dirty when an injection occurs. The antiseptic film 73 includes an adhesive for holding it in place. Once an injection has occurred, the punctured antiseptic film 73 is removed and replaced with a new antiseptic film 73.

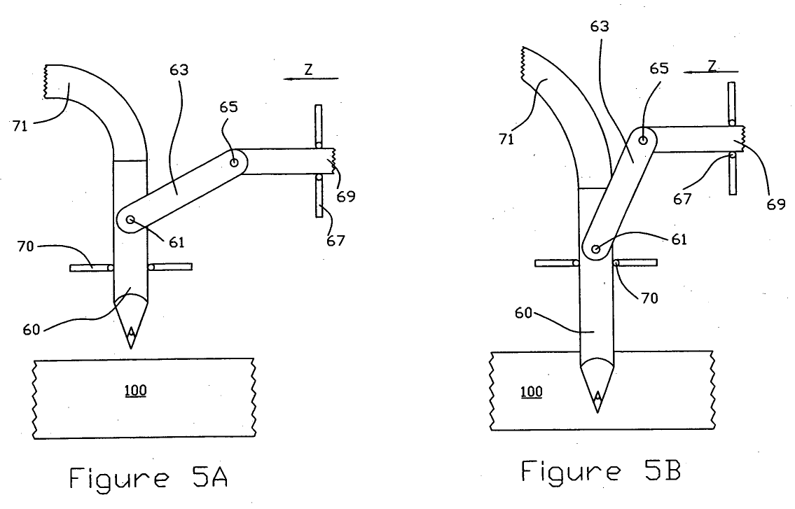

FIGS. 5A and 5B are perspective views of a needle 60 in different positions. In FIG. 5A, the needle 60 is shown in a retracted or ready-to-use position. FIG. 5B depicts the needle 60 after it has penetrated the skin 100 of the user. An injection may be electronically given when the microprocessor 51 determines that an adverse medical condition has arisen. Alternatively, the user may push a manual medication activation button 29 to manually inject stored medication.

In the preferred embodiment, a driving member 69 connects at one end to the manual medication activation button 29 and at an opposite end to a needle member 63. Lateral force exerted in the direction of arrow Z forces the needle 60 downward when the manual medication activation button 29 is pushed. The driving member 69 and needle member 63 are connected via pivot point 65. The needle member 63 is connected to the needle 60 via pivot point 61, as shown.

Guides 67 and 70, ensure that the needle 60 and the driving member 69 properly align, for smooth operation of the injection process. However, it is contemplated that other types of devices may be utilized to convert a lateral or horizontal force into a vertical force for driving the needle 60 into the skin of the user. An end of the needle 60 connects to a capsule puncture 77 via a tube 71.

It is contemplated that the driving member 69 is connected to, or alternatively, part of a plunger for a solenoid. Using an appropriate power source, the solenoid is energized by a control signal from the microprocessor. However, it should be noted that either the driving member 69 or the needle 60 might be incorporated into a design of a solenoid. For example, the driving member 69 may be part of a plunger in the solenoid. Otherwise, the needle 60 or driving member 69 may be actuated with a piezoelectric or magnetostrictive member that drives the needle 60 or driving member 69. An end or side of the piezoelectric or magnetostrictive member is secured to the casing 21 and arranged such that the member flexes or stretches in a specific direction. This flexing motion is harnessed to drive the needle into the skin of the user.

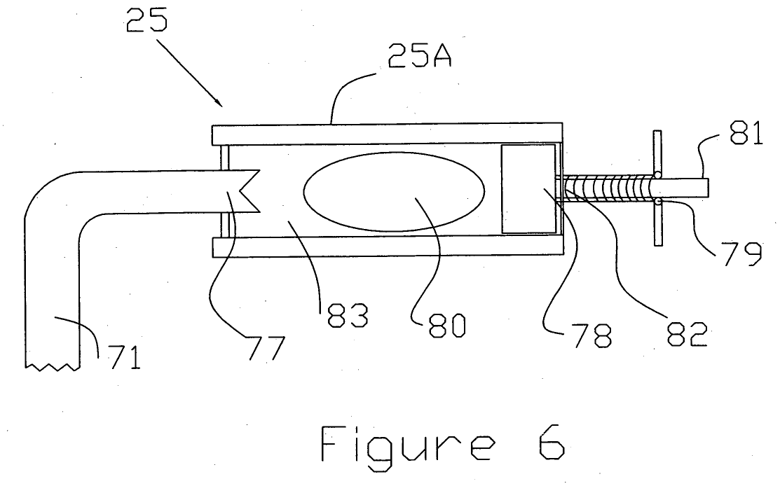

FIG. 6 is a perspective view of the medication capsule piercing device 77. Movement from the manual medication activation button 29, or electronic activation means that drives the needle 60, is translated to a plunger end 78 that pushes a medication capsule 80 into a capsule puncture 77 and forces medication from the capsule into a tube 71 having an opposite end connected to a needle 60. The needle 60 is arranged to puncture the skin just prior to puncturing the capsule 80. A return spring 82 withdraws the needle 60 from the user's skin after the manual medication activation button, or alternatively the electronic activation means, is released. The spent medication capsule 80 is removed and replaced with a new medication capsule 80.

Operation of the System

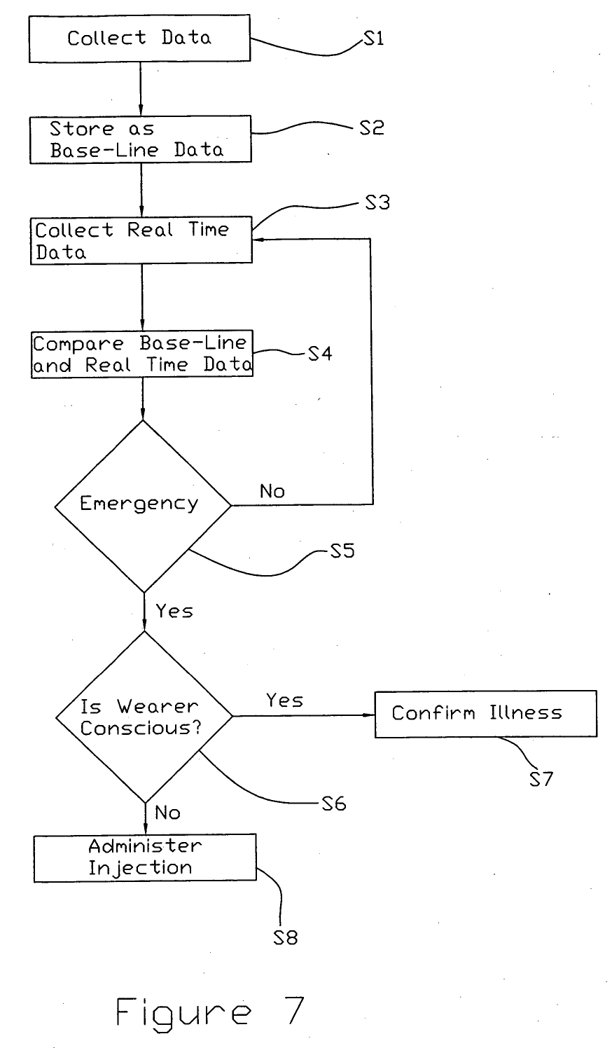

FIG. 7 is a flowchart of the preferred monitoring process of the remote monitoring unit. In step S1, the remote monitoring unit collects physiological data relating to the health of the wearer. This collected data is stored as base-line data in step S2. Periodically, this data is updated to correspond with changes in physical health conditions created by changing treatment techniques, losing weight and varying medications. Typically, this base-line data is recorded during a variety of ordinary tasks such that a pattern may be established.

In steps S3 and S4, real time data is periodically collected and compared to the stored base-line data. When the real time data deviates greatly from the base-line data, the system recognizes a medical condition or emergency in step S5. The system reviews the sensor information to identify the type of medical condition that exists. That is to say, a heart attack victim may experience a racing heart and profuse sweating; whereas, an insulin shock victim may sweat profusely while the pulse rate drops dramatically. If no medical condition is recognized, then the remote monitoring unit returns to step S3 and continues to periodically collect and compare real time data with the stored base-line data.

If the remote monitoring unit detects an emergency, a user is cued to confirm whether he is conscious in step S6. If conscious, the user is requested to confirm the medical condition in S7. If no response is received from the user, the system automatically administers an injection in step S8. If the user is conscious he may be provided a list of medical instructions to follow including confirming the existence of the medical condition. If the user is unconscious, then the system determines the appropriate medication to administer in accordance with the identified medical condition.

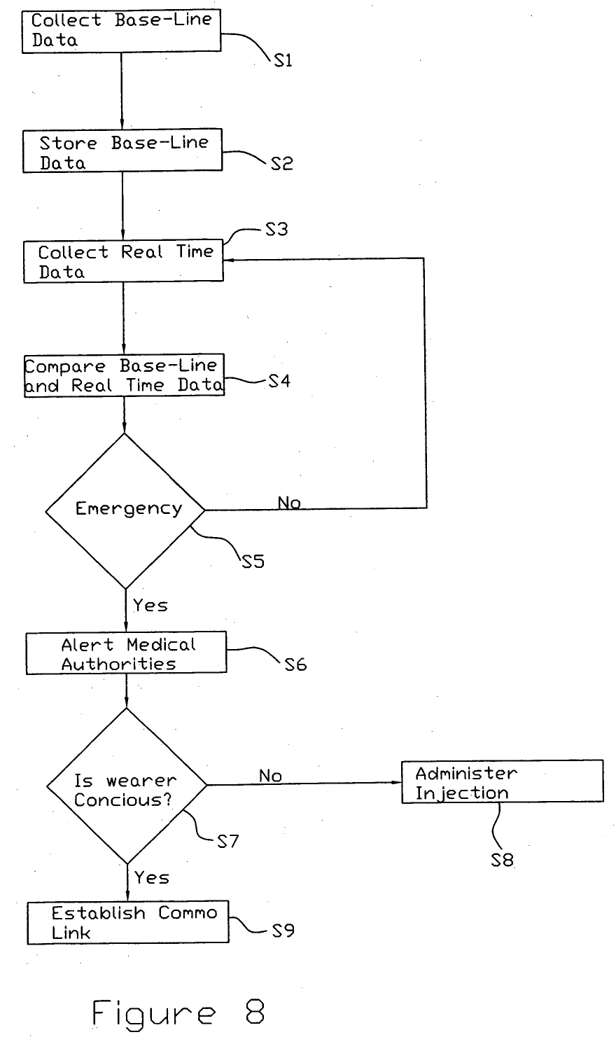

FIG. 8 is a flowchart of the preferred monitoring process of the central monitoring unit. It should be noted that either the remote monitoring unit or the central monitoring unit might control injection of the medication. The first steps, S1-S5, of the process parrot those of the remote monitoring unit.

In the preferred embodiment, the central monitoring unit alerts the medical authorities after an emergency has been recognized in step S6. The central monitoring then determines whether the user is conscious and either establishes a communications link or administers the appropriate medication in steps S7-S9.

While the invention has been described with respect to preferred embodiments, it is apparent to those skilled in the art that changes, modifications and additions may be made to the herein described embodiments without departing from the scope of the invention. Accordingly, it is intended that all matter contained in the above description or shown in the accompanying drawings shall be interpreted as illustrative and not in limiting sense or use.

1. A monitoring system that monitors physiological data of a wearer that includes a central monitoring unit and a remote monitoring unit, said remote monitoring unit comprising:
a microprocessor that compares collected real time physiological data with stored base-line physiological data to determine the existence of a medical condition of a wearer;
a memory including instructions for collecting and storing said real time and base-line physiological data relating to a health condition of a wearer;
a display connected to said microprocessor for displaying instructions for use of the remote monitoring unit;
at least one sensor connected to said microprocessor that periodically samples said physiological data;
at least one medication storage compartment for storing a medication to be administered in response to a medical condition;
an injection device connected to said at least one medication storage for delivering an injection of said medication to the wearer when the medical condition is detected; and,
a communications device that transmits collected physiological data from the remote monitoring unit to the central monitoring unit.

2. The monitoring system of claim 1 further comprising a global positioning system receiver that detects a location of the remote monitoring unit, such that said location of the remote monitoring unit may be relayed to the central monitoring unit.

3. The monitoring system of claim 1 wherein said communications device includes a speaker and microphone for providing audio communications between the wearer and an operator located at the central monitoring unit.

4. The monitoring system of claim 1 wherein said communications device includes a speaker, microphone and camera for providing audio and video communications between the wearer and an operator located at the central monitoring unit.

5. The monitoring system of claim 1 further comprising a needle compartment for housing said injection device and including an antiseptic film having a side exposed to the injection device and an opposite side exposed to a surface of a wearer's skin such that a needle included in said injection device penetrates and punctures said antiseptic film when the medication is administered.

6. The monitoring system of claim 1 wherein said injection device includes a solenoid for remotely administering the medication.

7. The monitoring system of claim 1 wherein said injection device includes a piezoelectric member for driving the injection device.

8. The monitoring system of claim 1 wherein said injection device includes a magnetostrictive member for driving the injection device.

9. The monitoring system of claim 1 wherein said medication storage compartment comprises a hinged cover.

10. The monitoring system of claim 1 wherein said remote monitoring unit comprises a speaker that alerts nearby individuals to a medical condition of a wearer when the wearer is unconscious.

11. The monitoring system of claim 1 further comprising a re-transmitter unit that relays information and data between the remote monitoring unit and the central monitoring unit.

12. A medical condition monitoring system that includes a central monitoring unit and a plurality of remote monitoring units, said system comprising:
the central monitoring unit that includes a transceiver for communicating with the remote monitoring unit;
a microprocessor within the central monitoring unit for storing physiological data of a plurality of wearers and for comparing collected real time physiological data with stored base-line physiological data to determine the existence of a medical condition;
a display screen connected to said central monitoring unit for displaying information from a selected remote monitoring unit;
a microprocessor located within each remote monitoring unit for comparing collected real time physiological data with stored base-line physiological data to determine the existence of a medical condition of a wearer;
a memory within each remote monitoring unit including instructions for collecting and storing said real time and base-line physiological data relating to a health condition of a wearer;
a display connected to said microprocessor located within each remote monitoring unit for displaying instructions for use of the remote monitoring unit;
at least one sensor located within each remote monitoring unit and connected to said microprocessor for periodically sampling physiological data of a wearer;
at least one medication storage compartment located within each remote monitoring unit for storing a medication to be administered in response to a detected medical condition;
an injection device located within each remote monitoring unit and connected to said at least one medication storage for delivering an injection of said medication to the wearer when the medical condition is detected; and,
a communication device located within each remote monitoring unit that transmits collected physiological data from the remote monitoring unit to the central monitoring unit.

13. A process for monitoring a wearer of a health monitoring device comprising:
collecting data during normal activities and when a wearer is not experiencing a serious medical condition;
storing said data as base-line data;
collecting and comparing real time data with said base-line data;
recognizing the existence of a medical condition when said real time data exceeds an acceptable threshold; and,
administering an injection of medication when said medical condition is recognized.

14. The process of claim 13 further comprising:
confirming consciousness with said wearer to determine whether an injection should be automatically administered.

15. The process of claim 13 further comprising:
providing a list of medical instructions to said wearer.

16. The process of claim 13 further comprising alerting medical authorities when a medical condition has been determined.
